(19) United States
(12) Patent Application Publication
(10) Pub. No.: US 2021/0275269 A1
(43) Pub. Date: Sep. 9, 2021

(54) MAGNETIC PROPULSION MECHANISM FOR MAGNETIC DEVICES

(71) Applicant: BIONAUT LABS LTD., Herzliya (IL)

(72) Inventors: Michael SHPIGELMACHER, Los Angeles, CA (US); Alexander SROMIN, Ashdod (IL)

(73) Assignee: BIONAUT LABS LTD., Herzliya (IL)

(21) Appl. No.: 17/258,407

(22) PCT Filed: Jul. 11, 2019
(86) PCT No.: PCT/US2019/041309

§ 371 Date: Jan. 6, 2021

Related U.S. Application Data
(60) Provisional application No. 62/697,044, filed on Jul. 12, 2018.

(51) Int. Cl. A61B 34/00

(52) U.S. Cl. CPC A61B 34/73; A61B 2034/731

(57) ABSTRACT

This invention relates to apparatus for propelling an internal device within a viscous medium in a biological tissue. The invention relates to a device, an apparatus and to systems and methods for device propulsion and for medical use of the propelled device.

### FIELD OF THE INVENTION

This invention relates to apparatus for propelling an internal device within a viscous medium in a biological tissue. The invention relates to a device, an apparatus and to systems and methods for device propulsion and for medical use of the propelled device.

### BACKGROUND OF THE INVENTION

In many medical applications, it can be useful to use a mobile medical device to move in a living organism. For example, it may be desirable to move an internal device through tissue to a particular anatomic location to release a drug, gather diagnostic data, or conduct a remote controlled surgical operation. To obtain such movement, propulsion modes utilizing magnetic fields have been developed.

One propulsion mode involves applying an external uniform rotating magnetic field on internal device located inside the body. According to this mode, the internal device has a helical shape (screw-like) and it comprises an embedded magnet with diametric magnetization. Rotating the external field exerts rotational torque on the device, propelling it forward (like a screw). This propulsion method is denoted as “rotation.”

Another propulsion mode involves applying an external non-uniform magnetic field (a magnetic gradient) on an internal device located inside the body. The device comprises an embedded magnet or a metallic component. In response to the external field gradient, the device would move along the gradient lines generated by the external magnet. This propulsion method is denoted as “gradient-based motion.”

Each propulsion technique has limited abilities in view of the motion schemes required for internal devices traveling within a tissue.

### SUMMARY OF THE INVENTION

The current invention provides devices and methods that combine both rotation and gradient-based motion for the propulsion of internal devices in a variety of combinations.

In one embodiment, this invention provides an apparatus for propelling an internal device within a viscous medium in a biological tissue, from a first location in the biological tissue to a second location in the biological tissue, the apparatus comprising:
a first external magnetic source (S1) outside of the biological tissue, the external magnetic source operative to generate a rotatable non-uniform magnetic field having an axis of rotation substantially along a line from the internal device to the external magnetic source, the non-uniform magnetic field having flux lines substantially orthogonal to the axis of rotation;
a local magnet (M1) in the internal device, the local magnet having a local magnetic field with flux lines substantially orthogonal to the axis of rotation; and
at least one helical projection on a surface of the internal device, the at least one helical projection interacting with the viscous medium to produce, from a rotation of the internal device, a rotation-based propulsive force on the internal device relative to the biological tissue;

wherein:
i. the non-uniform magnetic field of the external magnetic source (S1) is coupled to the local magnetic field of the local magnet (M1) of the internal device, such that a rotation of the non-uniform magnetic field of the external magnetic source (S1) on the axis of rotation produces a corresponding rotation of the local magnet (M1) and of the internal device;
ii. the non-uniform magnetic field has a substantial non-zero flux density gradient whose interaction with the local magnet of the internal device produces a gradient-based propulsive force substantially in a direction of the line from the internal device to the external magnetic source; and
iii. the rotation-based propulsive force combines with the gradient-based propulsive force to propel the internal device from the first location in the biological tissue towards the second location in the biological tissue.
In one embodiment, the first external magnetic source (S1) comprises a permanent magnet, an electromagnet or a combination thereof. In one embodiment, the first external magnetic source (S1) is a dipole magnetic source, and wherein the local magnet (M1) of the internal device is a dipole magnet. In one embodiment, the first external magnetic source (S1) is a flat permanent magnet, in the form of rectangle or a flat disk, magnetized across the flat surface.

In one embodiment, the permanent magnet comprises at least two magnets arranged in different magnetization directions, configured to maximize the magnetic flux in a specific plane and/or to maximize the magnetic gradient along a specific axis orthogonal to said plane. In one embodiment, the at least two magnets, are assembled in a non-planar orientation relative to each other, configured to maximize the magnetic flux in a specific plane and/or maximize the magnetic gradient along a specific axis orthogonal to said plane. In one embodiment, the assembly is constructed on a magnetic support structure (e.g., made of magnetic steel) to provide mechanical support to said assembly and to maximize the magnetic flux in a specific plane and/or to maximize the magnetic gradient along a specific axis orthogonal to said plane.

In one embodiment, the local magnet (M1) is a diametrically magnetized magnet.

In one embodiment the local magnet (M1) is located close to the front tip of the internal device (rather than closer to the tail of the internal device), to facilitate accurate control of the internal device motion due to the attraction of magnet M1 to the external magnetic source S1. It should be noted that if only one magnet M1 is used inside the internal device, and if the magnet M1 is placed close to the tail of the internal device, the tail may be attracted to the magnetic field generator S1, causing the internal device to twist (tail-first). Placing the magnet M1 closer to the front of the internal device prevents this problem.

In one embodiment, the internal device further comprises a magnetic component (M2), the magnetic component coupling magnetically with the non-uniform magnetic field of the external magnetic source (S1). In one embodiment, the magnetic component (M2) is axially magnetized along the axis of the helical projection.

In one embodiment, the apparatus further comprises a second external magnetic source (S2), said second source comprises a permanent magnet. In one embodiment, the magnetic component (M2), coupling magnetically with said second external source (S2).

In one embodiment, the magnetic component (M2) of the internal device comprises a magnetizable material, wherein the magnetizable material is coupled magnetically to the external first magnetic source, second magnetic source or a combination thereof.

In one embodiment, the magnetic component (M2) of the internal device comprises a permanent magnet with a magnetic field having flux lines substantially parallel to the line from the internal device to the external magnetic source.

In one embodiment, the non-uniform magnetic field (the field of S1) has a reversible flux direction; and
i. when the flux direction of the non-uniform magnetic field is a first direction along the line from the internal device to the external magnetic source, then the gradient-based propulsive force attracts the internal device toward the external magnetic source; and
ii. when the flux direction of the non-uniform magnetic field is a second direction along the line from the internal device to the external magnetic source, then the gradient-based propulsive force repels the internal device away from the external magnetic source.

In one embodiment, the permanent magnet (M2) comprises a spherical magnet (M2) that is free to rotate within a cavity in the internal device.

In one embodiment, the local magnet (M1) is partially enclosed within a magnetic shielding material. In one embodiment, the magnetic shielding material is removable and replaceable.

In one embodiment, this invention provides a method of propelling an internal device within a viscous medium in a biological tissue, from a first location in the biological tissue to a second location in the biological tissue, the method comprising:
a. providing (i) the internal device at the first location in the biological tissue and (ii) a first external magnetic source (S1) outside of the biological tissue;

wherein the internal device has an axis of rotation and comprises a local magnet (M1) having a local magnetic field with flux lines substantially orthogonal to the axis of rotation and at least one helical projection on a surface;
b. with the first external magnetic source (S1), generating a rotating non-uniform magnetic field having an axis of rotation substantially along a line from the internal device to the external magnetic source, the non-uniform magnetic field having flux lines substantially orthogonal to the axis of rotation; a local magnet (M1) in the internal device;

wherein rotation of the rotating non-uniform magnetic field of the external magnetic source (S1) on the axis of rotation produces a corresponding rotation of the local magnet (M1) and of the internal device, and from the rotation of the internal device the at least one helical projection interacts with the viscous medium to produce, a rotation-based propulsive force on the internal device relative to the biological tissue; and

wherein the rotating non-uniform magnetic field has a substantial non-zero flux density gradient whose interaction with the local magnet of the internal device produces a gradient-based propulsive force substantially in a direction of the line from the internal device to the external magnetic source; and

wherein the rotation-based propulsive force combines with the gradient-based propulsive force to propel the internal device from the first location in the biological tissue towards the second location in the biological tissue; and
c. adjusting the rotating non-uniform magnetic field to propel the internal device from the first location to the second location in the biological tissue.

In one embodiment, the first external magnetic source (S1) comprises a permanent magnet, an electromagnet or a combination thereof. In one embodiment, the first external magnetic source (S1) is a dipole magnetic source, and wherein the local magnet (M1) of the internal device is a dipole magnet. In one embodiment, the first external magnetic source (S1) is a flat permanent magnet, in the form of rectangle or a flat disk, magnetized across the flat surface.

In one embodiment, the permanent magnet comprises at least two magnets arranged in different magnetization directions, configured to maximize the magnetic flux in a specific plane and/or to maximize the magnetic gradient along a specific axis orthogonal to said plane. In one embodiment, the at least two magnets, are assembled in a non-planar orientation relative to each other, configured to maximize the magnetic flux in a specific plane and/or maximize the magnetic gradient along a specific axis orthogonal to said plane. In one embodiment, the assembly is constructed on a magnetic support structure (e.g., made of magnetic steel) to provide mechanical support to said assembly and to maximize the magnetic flux in a specific plane and/or to maximize the magnetic gradient along a specific axis orthogonal to said plane.

In one embodiment, the local magnet (M1) is a diametrically magnetized magnet.

In one embodiment, the internal device further comprises a magnetic component (M2), the magnetic component coupling magnetically with the rotating non-uniform magnetic field of the external magnetic source (S1). In one embodiment, the magnetic component (M2) is axially magnetized along the axis of the helical projection.

In one embodiment, the method further comprising providing a second external magnetic source (S2) outside of the biological tissue, said second source comprises a permanent magnet.

In one embodiment, the magnetic component (M2), coupling magnetically with said second external source (S2).

In one embodiment, the magnetic component (M2) of the internal device comprises a magnetizable material, wherein the magnetizable material is coupled magnetically to the external first magnetic source, second magnetic source or a combination thereof.

In one embodiment, the magnetic component (M2) of the internal device comprises a permanent magnet with a magnetic field having flux lines substantially parallel to the line from the internal device to the external magnetic source.

In one embodiment, of the method,
the non-uniform magnetic field (the field of S1) has a reversible flux direction; and
   i. when the flux direction of the non-uniform magnetic field is a first direction along the line from the internal device to the external magnetic source, then the gradient-based propulsive force attracts the internal device toward the external magnetic source; and
   ii. when the flux direction of the non-uniform magnetic field is a second direction along the line from the internal device to the external magnetic source, then the gradient-based propulsive force repels the internal device away from the external magnetic source.

In one embodiment, the permanent magnet (M2) comprises a spherical magnet (M2) that is free to rotate within a cavity in the internal device.

In one embodiment, magnet (M2) may have diametrical magnetization orthogonal to the symmetry axis of the internal device (similar to M1), but also be able to rotate inside a cavity in the internal device in a given plane of rotation. For instance, this can be achieved by placing a diametrically magnetized magnet M2 (a cylinder) inside a cylindrical cavity of the same dimensions as M2, but without affixing it to the cavity. In such a configuration, M2, may exert a yaw torque on the internal device, correcting the direction of motion of the internal device if it is not aligned with the symmetry axis of the externally applied rotating magnetic field.

In one embodiment, magnet (M2) may have diametrical magnetization orthogonal to the symmetry axis of the internal device (similar to M1), but also be able to rotate inside a cavity in the internal device in a given plane of rotation. For instance, this can be achieved by placing a diametrically magnetized magnet M2 (a cylinder) inside a cylindrical cavity of the same dimensions as M2, but without affixing it to the cavity. In such a configuration, M2, may exert a yaw torque on the internal device, correcting the direction of motion of the internal device if it is not aligned with the symmetry axis of the externally applied rotating magnetic field.

In one embodiment, the local magnet (M1) is partially enclosed within a magnetic shielding material. In one embodiment, the magnetic shielding material is removable and replaceable.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates external magnetic sources utilized in embodiments of the invention; FIG. 1A. left shows an internal device with embedded magnet, magnetized across diameter, rotating in externally applied rotating magnetic field. The external magnetic field is generated by a flat permanent magnet, magnetized along flat surface, rotating in plane of magnetization (plane of flat surface); FIG. 1A. right is a side view corresponding to FIG. 1A, with magnetic flux lines. Note flux lines becoming denser closer to the permanent magnet; FIG. 1B. is a side view showing the magnetic flux lines for a magnet configuration comprising more than one magnet. Note that the flux is expected to be stronger vs. a simple configuration with magnetization along the plane of a magnetic block, as seen in FIG. 1A; FIG. 1C. is a side view of an additional configuration comprising more than one magnet.

FIG. 2 illustrates embodiments of an internal device comprising internal magnets; FIG. 2A is a side cross section of an internal device with magnets M1 and M2. M2 is magnetized along the long axis of the internal device; FIG. 2B is a bottom cross section of an internal device showing magnetization of M1 across diameter; FIG. 2C is a 3D image of an internal device with magnets M1/M2 embedded inside (not visible).

FIG. 3 Illustrates an apparatus configuration comprising an internal device D1; a first external source S1; a second external source S2. D1 comprises embedded magnet/multiple magnets; Magnetic field source S1 generates rotating field; Magnetic field source S2 generates field gradient along axis of S1 rotation. FIG. 3A shows S1, S2 and D1; FIG. 3B is a side view of the apparatus of FIG. 3A, showing magnetic flux lines.

FIG. 4 illustrates an internal device D1 comprising M1 and M2; FIG. 4A is a side cross section of D1, showing M2 as freely-spinning magnetized sphere in a cavity; the Figure shows the cavity in which M2 spins freely, in response to rotating magnetic field around D1's long axis, a field generated by S1 (not shown). As M2 spins freely in the cavity it exerts no force or torque on device D1; FIG. 4B illustrates a device D1 under the influence of external field S2. Under the influence of a gradient generated by S2 along the D1 long axis, M2 starts pushing against the cavity wall, helping propel D1 forward towards S2.

FIG. 5 illustrates embodiments of the internal device; FIG. 5A. is a side cross section of an internal device with magnets M1, M2; FIG. 5B. is a bottom cross section of an internal device showing magnetization of M1 across diameter; FIG. 5C. is a 3D image of magnet M1, grey areas indicate placement of steel shielding minimizing flux orthogonal to plane of magnetization (generated by S2). Note that in this example shielding is symmetrical on both flat faces of M1 and partially covering opposing curved surfaces.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

i. Rotational Torque and Gradient-Based Motion:

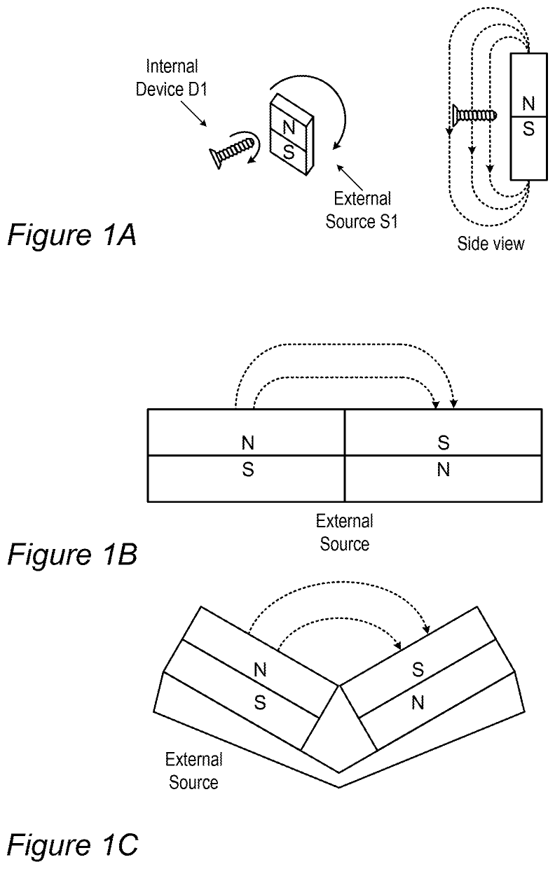

Assume the structure of a screw-like device with an embedded diametrically magnetized magnet. The “rotation” propulsion method simply applies a uniform magnetic field around the device. The device rotates as it seeks to align with the uniform field. Much effort is done by practitioners of this technique to generate a truly uniform rotating magnetic field (e.g., using Helmholtz or Maxwell coils). However, rather than generating a uniform rotating magnetic field, it was found that a non-uniform rotating magnetic field can be generated with a gradient component orthogonal to the plane of rotation, and this can enhance motion of the device. As an example, consider a rotating flat permanent magnet, such as a flat rectangle or a flat disk, magnetized across the flat surface (see external source S1 in FIG. 1A). As it rotates around an axis orthogonal to the flat surface, it generates a non-uniform magnetic field parallel to the flat surface. The magnetic flux lines are denser close to the magnet surface. If the screw central axis of the device is identical to the axis of rotation of magnetic field, it is subjected to 2 modes of propulsion:

1. Rotational torque exerted by the alignment with the rotating magnetic field

2. Gradient-based motion along the central axis towards the large rotating magnet.

Note that the 2 components are in the same direction (assuming the rotation direction matches the screw chirality). Hence, in contrast to only using one of propulsion modes, two propulsion modes are used, and such use improves device mobility. This is analogous to pushing AND rotating a screw vs. only rotating or only pushing it (see FIG. 1A).

Another advantage of such a setup is the fact that it requires a rotating fixed magnet only on one side of the device (not enclosing the volume, allowing easier access to patient). This allows using it on large subjects (e.g., large animals or humans) more easily. For reference, generating a uniform magnetic field using Helmholtz coils or permanent magnets across a large volume (like the body of a human patient) is notoriously difficult and such systems are very heavy and require complex engineering.

It should be noted that said non-uniform rotating magnetic field (with a gradient) can be generated using other methods, such as combinations of electromagnetic coils or permanent magnets (not only the spinning permanent magnet configuration described above).

It should also be noted that it is possible to shift the direction of the non-uniform rotating magnetic field as well as the axis of rotation and direction of the gradient (e.g., by shifting the rotating permanent magnet), thereby changing the direction of device motion.

ii. Device Comprising Additional Magnetic Component:

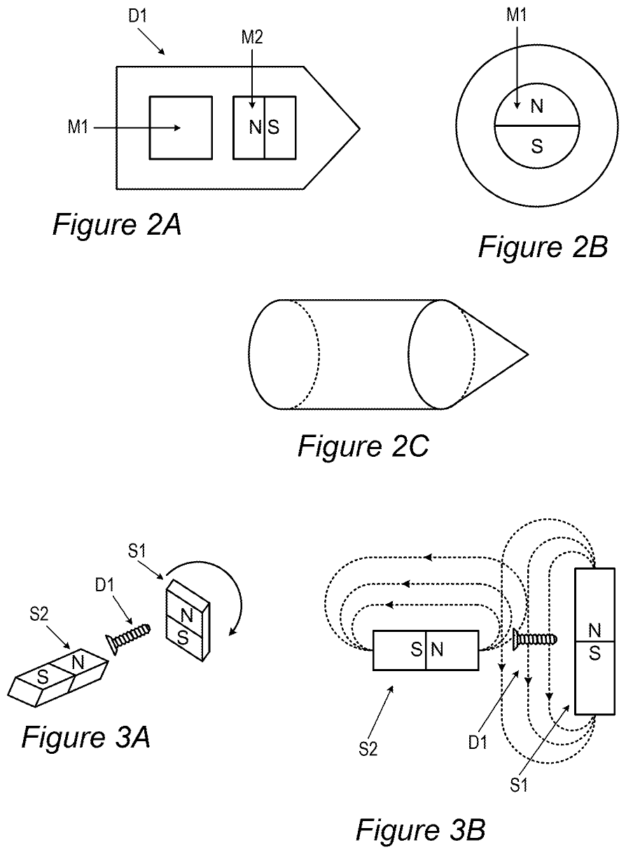

It should be noted that the gradient-based motion can be further enhanced by adding another magnetic component to the device. Denoting the diametrically magnetized magnet inside the device as M1 (diametrical magnetization means across the diameter of the propelling device). Define the new magnetic component inside the device as M2 (see FIG. 2A). Such magnetic component M2 would not be diametrically magnetized (unlike the first magnet M1, which enables rotational propulsion). Instead, M2 is axially magnetized in the direction of motion of the screw. Hence, it is subjected to greater attraction force by the gradient, as described above, further increasing mobility. Note that the location of the two magnets M1, M2 in the device can be designed in different ways (e.g., M2 in the tail, M2 in the head, vice versa, or other configurations). Also, note that the M1/M2 can have different shapes (e.g., rod, cube, disk, etc.).

iii. Device Comprising Additional External Source:

As described herein above, a source of rotating magnetic field S1 is provided. An additional external magnet/source of magnetic field (S2) can be added to increase the magnetic field gradient operating on magnet M2 as defined above. For instance, an external large permanent magnet S2 can be placed, which is magnetized along the axis of propulsion of the device, orthogonal to the rotating magnetic field (see FIG. 3A, 3B). It therefore exerts push/pull gradient-based force (depending on the direction of magnetization of M2, S2) on magnet M2, further assisting propulsion. Note that this external magnet S2 can be permanent or an electromagnet. Note that it is possible to switch this gradient component on or off by the following methods (among others):

For an electromagnet, it can be switched on an off;

For a permanent magnet, it can be fitted with a steel shield to redirect the flux away from the device when not needed.

Such on/off switch mechanism may be needed to prevent interference between the rotational component (generated by S1, affecting magnet M1) and the gradient component (generated by S2, affecting magnetic component M2). For instance, it is possible to apply the rotational component, switch it off and then apply the axial gradient, in a repeated fashion.

It should be noted that because M1 and M2 are orthogonal to each other, they are expected to interact with orthogonal field components (rotating field, gradient) separately with little interference.

iv. Magnetic Component M2

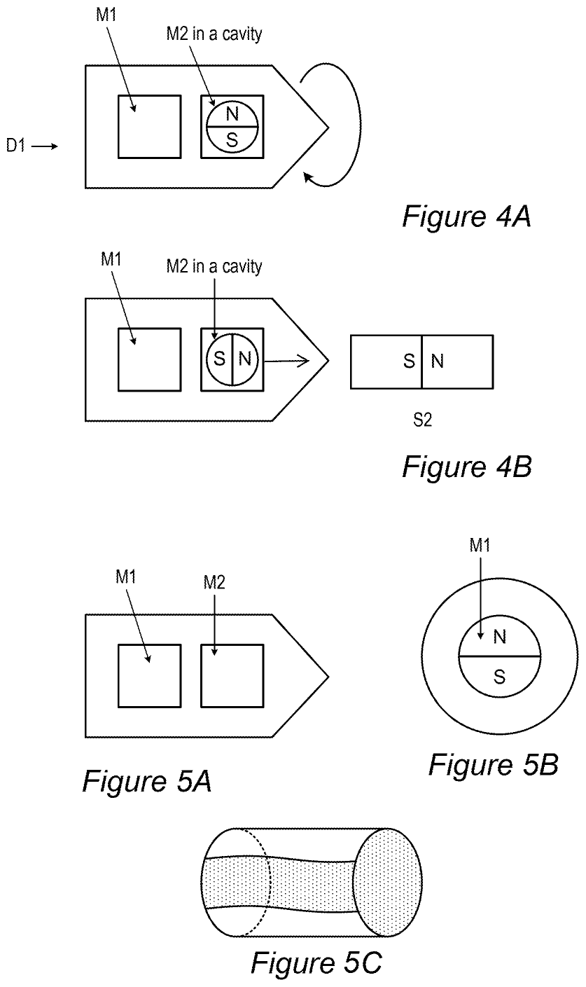

It should be noted that for a system comprising M1/M2 and S1/S2 as described above there could still be interaction between magnet M2 and the rotational magnetic field generated by S1, which may result in unnecessary torque affected on the device by M2 as it tries to align with the rotating field (instead of its position along the axis of rotation). One way to remedy this is by designing magnet M2 as a sphere, or another shape freely rotating in a cavity inside the device (see FIG. 4A, 4B). This means that it does not generate any torque on the device in response to the rotating magnetic field S1 (as it spins freely). However, when the sphere is subjected to a push/pull gradient applied by magnetic field source S2, it is pushed/pulled and exerts the corresponding force on the device, propelling it in the right direction. In some embodiments, lubricants or other coating inside the cavity are added to allow for free rotation of the magnet M2. Another option to achieve the same effect is by having M2 connected to a rotational axis or gimbals inside the cavity so it can spin freely generating no torque or force in response to the rotating magnetic field generated by S1.

v. Magnetic Shielding Component M2

It should be noted that in the embodiments described herein above, there could still be interaction between magnet M1 and the magnetic field gradient generated by S2, which may result in unnecessary torque affected on the device by M1 as it tries to align with the flux lines of S2 along the axis of rotation (instead of the magnetic flux lines in the plane of rotation). A way to remedy this is by placing magnetic shielding material (e.g., steel) in a configuration shielding the field the component parallel to the axis of rotation (see FIG. 5C, an example of a shielding configuration for magnet M1). That way, magnet M1 will be effectively exposed only to the magnetic field component rotating in the plane of rotation and not to the component along the axis, thereby maintaining effective rotation in the correct direction. Note that this shielding of M1 is possible for any configuration employing M2 as described herein above.

### Devices of the Invention

In one embodiment, this invention provides internal devices capable of moving within a viscous medium. The term “internal device” means that the device is capable of being propelled within a certain medium, thus being internal to this medium. The internal viscous medium is a biological tissue in one embodiment.

In one embodiment, the device comprises local magnet (M1), the local magnet having a local magnetic field with flux lines substantially orthogonal to the axis of rotation. In one embodiment, the device comprises at least one helical projection on its surface. In one embodiment, the at least one helical projection interacts with the viscous medium to produce, from a rotation of the internal device, a rotation-based propulsive force on the internal device relative to the biological tissue. According to this aspect and in one embodiment, the device assumes a screw-like structure and in response to external magnetic field, the device rotates around it's long axis. The rotation results in propagation of the device in accordance with the direction of the helical projection.

In one embodiment, a non-uniform magnetic field of an external magnetic source (S1) is coupled to the local magnetic field of the local magnet (M1) of the internal device, such that rotation of the non-uniform magnetic field of the external magnetic source (S1) around the axis of rotation produces a corresponding rotation of the local magnet (M1) and of the internal device.

In one embodiment, the non-uniform magnetic field has a substantial non-zero flux density gradient whose interaction with the local magnet of the internal device produces a gradient-based propulsive force substantially in a direction of the line from the internal device to the external magnetic source.

In one embodiment, the rotation-based propulsive force combines with the gradient-based propulsive force to propel the internal device from the first location in the biological tissue towards the second location in the biological tissue. In one embodiment, the combination of the rotation-based propulsive force and the gradient-based propulsive force enhance propulsion of the device when compared to a single propulsion mechanism i.e. rotation-based propulsion or gradient-based propulsion alone.

In one embodiment, the local magnet (M1) of the internal device is a dipole magnet. In one embodiment, the local magnet (M1) is a diametrically magnetized magnet. According to this aspect and in one embodiment, the device has an elongated shape (e.g., rod, cone, screw, nail shape) and the diametrically magnetized magnet corresponds to the diameter(s) of the cross section of the long axis of the device.

In one embodiment, the device further comprises a magnetic component (M2). In one embodiment, the magnetic component couples magnetically with a non-uniform magnetic field of an external magnetic source (S1). In one embodiment, the magnetic component (M2) is axially magnetized along the axis of the helical projection. In one embodiment, the magnetic component is designed to be affected by a second external magnetic source (S2), S2 comprises a permanent magnet.

In one embodiment, the magnetic component (M2), couples magnetically with the second external source (S2). In one embodiment, the magnetic component (M2) of the internal device comprises a magnetizable material, wherein the magnetizable material is coupled magnetically to the external first magnetic source, second magnetic source or a combination thereof.

In one embodiment, the magnetic component (M2) of the internal device comprises a permanent magnet with a magnetic field having flux lines substantially parallel to the line from the internal device to the external magnetic source.

In one embodiment, the device responds to the external field as follows:

The non-uniform magnetic field (the field of S1) has a reversible flux direction; and
i. when the flux direction of the non-uniform magnetic field is a first direction along the line from the internal device to the external magnetic source, then the gradient-based propulsive force attracts the internal device toward the external magnetic source; and
ii. when the flux direction of the non-uniform magnetic field is a second direction along the line from the internal device to the external magnetic source, then the gradient-based propulsive force repels the internal device away from the external magnetic source.

In one embodiment, the device comprises a cavity. In one embodiment, the permanent magnet (M2) is free to rotate within a cavity in the internal device. In one embodiment, the free-rotating magnet comprises a spherical magnet (M2).

In one embodiment, the local magnet (M1) is partially enclosed within a magnetic shielding material. In one embodiment, the magnetic shielding material is removable and replaceable.

Materials: Devices of this invention are made from materials compatible with the device use. In some embodiments, the device is made of a biocompatible material. In one embodiment, the device is made of a bio-degradable material. In another embodiment, the device is made of a non-bio-degradable material. In one embodiment, the device comprises a magnet, a magnetic component or a combination thereof. In one embodiment, the magnetic component comprises a magnetic material. In one embodiment, the magnetic material comprises a ferromagnetic or a paramagnetic material. In some embodiments, the ferromagnetic material or the paramagnetic material is any ferromagnetic or paramagnetic material known in the art. In some embodiments the ferromagnetic material comprises Co, Fe, Ni, Gd, Tb, Dy, Eu, oxides thereof, alloys thereof or mixtures thereof. In some embodiments, the device comprises metals. In some embodiments, the metal comprises Fe, Ti, Ni, Co, Au, Ag, Cr, Al, Cu, Pt, Pd alloys thereof or combinations thereof. In some embodiments, the device comprises silicon. In some embodiments, the device comprises silicon oxide. In some embodiments, the device comprises organic materials. In some embodiments the device comprises polymers. In some embodiments, the polymer comprises Polydimethylsiloxane (PDMS), polystyrene (PS), PTFE, polyester, acrylate-based polymer, PVC or any other polymer as know n in the art. In some embodiments, the device comprises any combination of metals, metal alloys, metal oxides, III-IV materials, silicon, silicon oxide, organic materials, composite materials. In one embodiment, the external surface of the device or portions thereof is coated by a coating material. In one embodiment, the coating material is a low-friction material. In one embodiment, the thickness of the coating material ranges between 1 nm and 10 microns.

Shape and Geometry: Devices of this invention are formed in any shape suitable. In one embodiment, the devices comprise an external helical shape. In one embodiment, the devices are screw-like shaped. In one embodiment, the devices are of an elongated shape. In one embodiment, the device is shaped as a rod, a cone, a disc, a sphere, a pyramid, a box, a cylinder, a cube. In one embodiment, the device is symmetric. In one embodiment, the device is asymmetric. In one embodiment, the device comprises symmetric and asymmetric portions. In one embodiment, the device is tapered along a certain direction. In one embodiment, the device comprises a sharp end, a blunt end or a combination thereof. Any shape or geometry may be suitable for devices of this invention. In some embodiments, the devices comprise a cavity. In one embodiment, the cavity volume is less than 50% of the total device volume. In another embodiment, the cavity volume is more than 50% of the device total volume. In one embodiment, the device comprises more than one cavity.

Dimensions: In some embodiments, devices of this invention are micro-devices. In some embodiments, devices of this invention are nano-devices. In some embodiments, devices of this invention are characterized by having dimensions in the micrometer range and dimensions in the nanometer range. Micro-devices are devices having at least one dimension in the micrometer range i.e. between 1 micron and 1000 micron. Nanodevices are devices having at least one dimension at the nanometer range i.e. between 1 nm and 1000 nm. In some embodiments, devices of this invention are characterized by dimensions in the mm range or in the cm range. Devices of this invention may be characterized by having a combination of dimensions, e.g., having dimensions in the cm, mm, micrometers and in the nanometers range. In one embodiment, the largest dimension of the device is in the micron range, and the device comprise features measuring in the micrometer and/or in the nanometer range. In one embodiment, micro-devices are devices wherein the largest dimension is in the micrometer range. In one embodiment, nanodevices are devices wherein the largest dimension is in the nanometer range.

In some embodiments, the devices, the magnetic components, the magnets or a combination thereof comprise ferromagnetic and/or paramagnetic components/materials. In some embodiments, the devices or components within the devices are flexible. In some embodiments, the devices or components within the devices are non-flexible. In some embodiment, the devices or components within the devices are water repellant. In some embodiments, the devices or components within the devices are porous.

In one embodiment, in addition to the magnetic feature(s) included in the device for propulsion, the device further comprises elements designed to perform medical tasks as detailed herein below. Such elements include but are not limited to:
i. cavities, pores or compartments in the device or on the surface of the device for holding pharmaceutical ingredients/drugs, or other materials, chemical compounds or biological compounds;
ii. elements used for drug (or other material) delivery such as open/close mechanism and components for opening and closing the drug-carrying cavity, pressure-inducing elements or other injectors to release the drug (material) from the cavity, actuators, sensors, timers, membranes, springs, seals etc.
iii. operation tools for sampling a tissue or for performing a medical procedure such as knife, blade, screw, spring, capturing devices such as tweezers, sharp tools, arrays of operating tools, syringes, suction elements, etc.
iv. sensors and elements responsive to external stimuli, e.g., US, magnetic or radiation responsive elements used to enable device imaging or used for start/stop of an action performed by the device.
v. elements for controlling the performance of a medical task, such as electronic circuits, electronic components, micro-electro-mechanical (MEMS) devices, remote-controlled elements etc.
vi. elements such as handles or other features to facilitate injection and retrieval of the devices to/from the biological tissue.

### Apparatuses and Systems of the Invention

In one embodiment, this invention provides an apparatus for propelling an internal device within a viscous medium in a biological tissue, from a first location in the biological tissue to a second location in the biological tissue. In one embodiment, an apparatus of this invention comprising an internal device capable of moving within a tissue and an external source capable of controlling the movement of the internal device. By “external” it is meant the that the source is external to the device, or that the source is external to the device and to the tissue.

In one embodiment, this invention provides an apparatus for propelling an internal device within a viscous medium in a biological tissue, from a first location in the biological tissue to a second location in the biological tissue, the apparatus comprising:
a first external magnetic source (S1) outside of the biological tissue, the external magnetic source operative to generate a rotatable non-uniform magnetic field having an axis of rotation substantially along a line from the internal device to the external magnetic source, the non-uniform magnetic field having flux lines substantially orthogonal to the axis of rotation;
a local magnet (M1) in the internal device, the local magnet having a local magnetic field with flux lines substantially orthogonal to the axis of rotation; and
at least one helical projection on a surface of the internal device, the at least one helical projection interacting with the viscous medium to produce, from a rotation of the internal device, a rotation-based propulsive force on the internal device relative to the biological tissue;
   wherein:
i. the non-uniform magnetic field of the external magnetic source (S1) is coupled to the local magnetic field of the local magnet (M1) of the internal device, such that a rotation of the non-uniform magnetic field of the external magnetic source (S1) on the axis of rotation produces a corresponding rotation of the local magnet (M1) and of the internal device;
ii. the non-uniform magnetic field has a substantial non-zero flux density gradient whose interaction with the local magnet of the internal device produces a gradient-based propulsive force substantially in a direction of the line from the internal device to the external magnetic source; and
iii. the rotation-based propulsive force combines with the gradient-based propulsive force to propel the internal device from the first location in the biological tissue towards the second location in the biological tissue.

In one embodiment, the first external magnetic source (S1) comprises a permanent magnet, an electromagnet or a combination thereof.

In one embodiment, the first external magnetic source (S1) is a dipole magnetic source, and wherein the local magnet (M1) of the internal device is a dipole magnet.

In one embodiment, the first external magnetic source (S1) is a flat permanent magnet, in the form of rectangle or a flat disk, magnetized across the flat surface.

In one embodiment, the permanent magnet comprises at least two magnets arranged in different magnetization directions, configured to maximize the magnetic flux in a specific plane and/or to maximize the magnetic gradient along a specific axis orthogonal to said plane.

In one embodiment, the at least two magnets, are assembled in a non-planar orientation relative to each other, configured to maximize the magnetic flux in a specific plane and/or maximize the magnetic gradient along a specific axis orthogonal to said plane.

In one embodiment, the assembly is constructed on a magnetic support structure (e.g., made of magnetic steel) to provide mechanical support to said assembly and to maximize the magnetic flux in a specific plane and/or to maximize the magnetic gradient along a specific axis orthogonal to said plane.

In one embodiment, the local magnet (M1) is a diametrically magnetized magnet.

In one embodiment, the internal device further comprises a magnetic component (M2), the magnetic component coupling magnetically with the non-uniform magnetic field of the external magnetic source (S1).

In one embodiment, the magnetic component (M2) is axially magnetized along the axis of the helical projection.

In one embodiment, the apparatus further comprising a second external magnetic source (S2), said second source comprises a permanent magnet.

In one embodiment, the magnetic component (M2), coupling magnetically with said second external source (S2).

In one embodiment, the magnetic component (M2) of the internal device comprises a magnetizable material, wherein the magnetizable material is coupled magnetically to the external first magnetic source, second magnetic source or a combination thereof.

In one embodiment, the magnetic component (M2) of the internal device comprises a permanent magnet with a magnetic field having flux lines substantially parallel to the line from the internal device to the external magnetic source.

In one embodiment, the non-uniform magnetic field (the field of S1) has a reversible flux direction; and
i. when the flux direction of the non-uniform magnetic field is a first direction along the line from the internal device to the external magnetic source, then the gradient-based propulsive force attracts the internal device toward the external magnetic source; and
ii. when the flux direction of the non-uniform magnetic field is a second direction along the line from the internal device to the external magnetic source, then the gradient-based propulsive force repels the internal device away from the external magnetic source.

In one embodiment, the permanent magnet (M2) comprises a spherical magnet (M2) that is free to rotate within a cavity in the internal device.

In one embodiment, the local magnet (M1) is partially enclosed within a magnetic shielding material. In one embodiment, the magnetic shielding material is removable and replaceable.

### Methods of the Invention

In one embodiment, this invention provides a method of propelling an internal device within a viscous medium in a biological tissue, from a first location in the biological tissue to a second location in the biological tissue.

In one embodiment, this invention provides a method of propelling an internal device within a viscous medium in a biological tissue, from a first location in the biological tissue to a second location in the biological tissue, the method comprising:
a) providing (i) the internal device at the first location in the biological tissue and (ii) a first external magnetic source (S1) outside of the biological tissue;
   wherein the internal device has an axis of rotation and comprises a local magnet (M1) having a local magnetic field with flux lines substantially orthogonal to the axis of rotation and at least one helical projection on a surface;
b) with the first external magnetic source (S1), generating a rotating non-uniform magnetic field having an axis of rotation substantially along a line from the internal device to the external magnetic source, the non-uniform magnetic field having flux lines substantially orthogonal to the axis of rotation; a local magnet (M1) in the internal device;
   wherein rotation of the rotating non-uniform magnetic field of the external magnetic source (S1) on the axis of rotation produces a corresponding rotation of the local magnet (M1) and of the internal device, and from the rotation of the internal device the at least one helical projection interacts with the viscous medium to produce, a rotation-based propulsive force on the internal device relative to the biological tissue;
   wherein the rotating non-uniform magnetic field has a substantial non-zero flux density gradient whose interaction with the local magnet of the internal device produces a gradient-based propulsive force substantially in a direction of the line from the internal device to the external magnetic source; and
   wherein the rotation-based propulsive force combines with the gradient-based propulsive force to propel the internal device from the first location in the biological tissue towards the second location in the biological tissue; and
c) adjusting the rotating non-uniform magnetic field to propel the internal device from the first location to the second location in the biological tissue.

In one embodiment, the first external magnetic source (S1) comprises a permanent magnet, an electromagnet or a combination thereof. In one embodiment, the first external magnetic source (S1) is a dipole magnetic source, and wherein the local magnet (M1) of the internal device is a dipole magnet.

In one embodiment, the first external magnetic source (S1) is a flat permanent magnet, in the form of rectangle or a flat disk, magnetized across the flat surface.

In one embodiment, the permanent magnet comprises at least two magnets arranged in different magnetization directions, configured to maximize the magnetic flux in a specific plane and/or to maximize the magnetic gradient along a specific axis orthogonal to said plane. In one embodiment, the at least two magnets, are assembled in a non-planar orientation relative to each other, configured to maximize the magnetic flux in a specific plane and/or maximize the magnetic gradient along a specific axis orthogonal to said plane.

In one embodiment, the assembly is constructed on a magnetic support structure (e.g., made of magnetic steel) to provide mechanical support to said assembly and to maximize the magnetic flux in a specific plane and/or to maximize the magnetic gradient along a specific axis orthogonal to said plane.

In one embodiment, the local magnet (M1) is a diametrically magnetized magnet.

In one embodiment, the internal device further comprises a magnetic component (M2), the magnetic component coupling magnetically with the rotating non-uniform magnetic field of the external magnetic source (S1). In one embodiment, the magnetic component (M2) is axially magnetized along the axis of the helical projection.

In one embodiment, the method further comprising providing a second external magnetic source (S2) outside of the biological tissue, the second source comprises a permanent magnet.

In one embodiment, the magnetic component (M2), coupling magnetically with said second external source (S2). In one embodiment, the magnetic component (M2) of the internal device comprises a magnetizable material, wherein the magnetizable material is coupled magnetically to the external first magnetic source, second magnetic source or a combination thereof.

In one embodiment, the magnetic component (M2) of the internal device comprises a permanent magnet with a magnetic field having flux lines substantially parallel to the line from the internal device to the external magnetic source.

In one embodiment, the non-uniform magnetic field (the field of S1) has a reversible flux direction; and
i. when the flux direction of the non-uniform magnetic field is a first direction along the line from the internal device to the external magnetic source, then the gradient-based propulsive force attracts the internal device toward the external magnetic source; and
ii. when the flux direction of the non-uniform magnetic field is a second direction along the line from the internal device to the external magnetic source, then the gradient-based propulsive force repels the internal device away from the external magnetic source.

In one embodiment, the permanent magnet (M2) comprises a spherical magnet (M2) that is free to rotate within a cavity in the internal device. In one embodiment, the local magnet (M1) is partially enclosed within a magnetic shielding material. In one embodiment, the magnetic shielding material is removable and replaceable.

In one embodiment, methods of propulsion of the invention provides enhanced propulsion capabilities. In one embodiment, during propulsion of the device, the device is imaged by an external imaging system. In one embodiment, the external imaging system comprises ultra sound (US) source. In one embodiment, the external imaging system comprises magnetic resonance imaging (MRI). In one embodiment, the imaging system comprises X-ray based imaging. A combination of the imaging techniques described above is also applicable in embodiments of the invention.

In one embodiment, methods of this invention comprise employing more than one device. According to this aspect and in one embodiment, two or more devices are propelled in a biological tissue. Two or more devices can be propelled in the same direction. Two or more such devices can be identical or can possess different elements/features. Two or more such devices may be prepared such that different devices are equipped for different purposes. According to this aspect and in one embodiment, some devices in a group of propelled devices may contain drugs to be delivered, while others may contain medical operational tools to perform a procedure in the area where the drug is delivered to or in a different area. The devices in a group of devices can be operated in concert and can perform multiple tasks in parallel or as a sequence. Such operation of a fleet of devices enables multi-task treatment in some embodiments. Such operation is useful and advantageous for complex procedures, for highly-sensitive procedures, for treatments requiring high accuracy, for small-volume sampling, for painless or less painful procedures, for efficient delivery, for fast procedures, for low-cost procedures etc.

### Uses

In one embodiment, this invention provides methods of use of apparatuses of the invention. In one embodiment, methods of this invention of propelling a device within a viscous medium are used for medical purposes.

In one embodiment, use of apparatuses and methods of this invention includes delivering a drug or a pharmaceutical ingredient to a certain location within or adjacent to a biological tissue. According to this aspect and in one embodiment, the drug is loaded on to (e.g., within) the device and the device is propelled to certain location where the drug is needed. At this defined location, the device expels the drug as needed.

In one embodiment, use of apparatuses and methods of this invention includes obtaining a biological sample from a certain location within or adjacent to a biological tissue. According to this aspect and in one embodiment, the device is propelled to a certain location from which a biological sample should be obtained. The device comprises element(s) required to obtain a sample (e.g., cutting tools/storage cavity/syringe/tweezers etc.)

In one embodiment, use of apparatuses and methods of this invention includes sensing/evaluating/monitoring/quantifying a biological condition/parameter in a certain location within or adjacent to a biological tissue. According to this aspect and in one embodiment, the device is propelled to a certain location in which a biological condition/parameter is needed to be tested. The device comprises element(s) required to sense/evaluate the parameter (e.g., pH sensor, sensor for certain biological material, water sensor, heat sensor, mechanical sensor etc.).

In one embodiment, use of apparatuses and methods of this invention includes imaging or mapping a biological region in a certain location within or adjacent to a biological tissue. According to this aspect and in one embodiment, the device is propelled to a certain location of which imaging/mapping is required. The device comprises element(s) required to image/map the required region (e.g., sensor or sensor array suitable for sensing energy or material signals, transducers, sensor control units, etc.).

In one embodiment, use of apparatuses and methods of this invention includes cutting/slicing/puncturing/applying pressure to/apply suction or pull force to/grabbing/pushing a portion of a biological tissue in a certain location within or adjacent to the biological tissue. According to this aspect and in one embodiment, the device is propelled to a certain location in which a mechanical procedure/operation is needed. The device comprises element(s) required to perform the procedure/operation (e.g., cutting tools/blade/syringe/tweezers/energy source/etc.).

Apparatuses of this invention are useful in some embodiments for drug delivery, for diagnostics, for medical treatments, for surgery and for any other medical procedure required for a certain location within or adjacent to a biological tissue.

### Definitions

An internal device in the context of this invention is a device that is operated internally in a tissue or adjacent to a tissue. A helical projection refers to projection in the form or shape of a helix, such as the helical projection of a screw. A local magnet M1 refers to the magnet M1 located in the internal device.

An apparatus of the invention is also referred to as a system in some embodiments. In some embodiments, the apparatus is part of a system. In some embodiments, systems of the invention comprise an apparatus of the invention. In some embodiments, the apparatus comprises a system.

As used herein, the term “a” or “one” or “an” refers to at least one. The phrase “two or more,” as used herein, may be of any denomination, which will suit a particular purpose. The terms “about” or “approximately” or “substantially” may comprise a deviance from the indicated term of +1%, or in some embodiments, −1%, or in some embodiments, ±2.5%, or in some embodiments, ±5%, or in some embodiments, ±7.5%, or in some embodiments, ±10%, or in some embodiments, ±15%, or in some embodiments, ±20%, or in some embodiments, ±25%.

While certain features of the invention have been illustrated and described herein, many modifications, substitutions, changes, and equivalents will now occur to those of ordinary skill in the art. It is, therefore, understood that the appended claims are intended to cover all such modifications and changes as fall within the true spirit of the invention.

### EXAMPLES

### Example 1

### Rotational Torque and Gradient-Based Motion

A device (D1) comprising a helical projection and a diametric magnet is inserted into a tissue. A rotating permanent magnet (S1), magnetized across a surface is provided external to the tissue. As the external rotating magnet rotates around an axis orthogonal to its magnetized surface, it generates a non-uniform magnetic field parallel to that surface. The magnetic flux lines are denser close to the magnet surface. The screw (helical projection) central axis of the device is identical to the axis of rotation of magnetic field, and it is subjected to 2 modes of propulsion:

1. Rotational torque exerted by the alignment with the rotating magnetic field

2. Gradient-based motion along the central axis towards the large rotating magnet.

The two components are in the same direction (assuming the rotation direction matches the screw chirality). Hence, two propulsion modes are used, leading to improved device mobility.

As required, the direction of the non-uniform rotating magnetic field is shifted, as well as the axis of rotation and direction of the gradient (e.g., by shifting the rotating permanent magnet), thereby changing the direction of device motion.

### Example 2

### Device Comprising Additional Magnetic Component

A device (D1) comprising a helical projection and a diametric magnet (M1) as described in Example 1 further comprises another magnetic component (M2) in the form of a magnet or a magnetic material. The device is inserted into a tissue. M2 is not diametrically magnetized (unlike the first magnet M1). Instead, M2 is axially magnetized in the direction of motion of the screw (helix). Hence, it is subjected to greater attraction force by the gradient induced by the external magnet, further increasing mobility.

### Example 3

### Device Comprising Additional External Source

As described in Example 2, a device (D1) comprising a helical projection, a diametric magnet (M1) and another magnetic component (M2) in the form of a magnet or of a magnetic material is provided. The device is inserted into a tissue. A source of rotating magnetic field (S1) is provided. An additional external magnet/source of magnetic field (S2) is added to increase the magnetic field gradient operating on magnet (M2). (S2) is an external large permanent magnet which is magnetized along the axis of propulsion of the device, orthogonal to the rotating magnetic field formed by (S1), or (S2) is an electromagnet. The field formed by (S2) exerts push/pull gradient-based force (depending on the direction of magnetization of M2, S2) on magnet M2, further assisting propulsion. As required, the gradient component is switched on or off by the following methods (among others):

a. for the electromagnet, the current is switched on an off;
b. for a permanent magnet, the magnet is fitted with a removable steel shield to redirect the flux away from the device when not needed.

Such an on/off switch mechanism prevents interference between the rotational component (generated by S1, affecting magnet M1) and the gradient component (generated by S2, affecting magnetic component M2). As required, the rotational component generated by S1 is applied, then switched off followed by the axial gradient generated by S2, in a repeated fashion.

When M2 is a magnet, M1 and M2 are magnetically orthogonal to each other, and they are expected to interact with orthogonal field components (rotating field, gradient) separately with little interference.

### Example 4

### Magnetic Component M2

For a system comprising M1/M2 and S1/S2 as described in Example 3, there could still be interaction between magnet M2 and the rotational magnetic field generated by S1, which may result in unnecessary torque on the device by M2. To prevent or reduce this interaction, M2 is designed as a sphere, or another shape, such that it freely rotates in a cavity inside the device. Accordingly, it does not generate any torque on the device in response to the rotating magnetic field S1 (as it spins freely). However, when the sphere is subjected to a push/pull gradient applied by magnetic field source S2, it is pushed/pulled and exerts the corresponding force on the device, propelling it in the right direction. As required, lubricants or other coating inside the cavity are added to allow for free rotation of the magnet M2. In some devices to achieve the same effect M2 is connected to a rotational axis or gimbals inside the cavity so it can spin freely generating no torque or force in response to the rotating magnetic field generated by S1.

### Example 5

### Magnetic Shielding Component M2

In order to prevent or reduce the interaction between magnet M1 and the magnetic field gradient generated by S2 in an apparatus as described in Examples 3 and 4, which may result in unnecessary torque affected on the device by M1 as it tries to align with the flux lines of S2 along the axis of rotation (instead of the magnetic flux lines in the plane of rotation), shielding for M1 is provided. A magnetic shielding material (e.g., steel) is placed in a configuration shielding (redirecting) the field component parallel to the axis of rotation (see FIG. 5C, an example of a shielding configuration for magnet MU The magnet M1 is thus effectively exposed only to the magnetic field component rotating in the plane of rotation and not to the component along the axis, thereby maintaining effective rotation in the correct direction. Note that this shielding of M1 is possible for any configuration employing M2 as described in Examples 2-4.

What is claimed is:

1. Apparatus for propelling an internal device within a viscous medium in a biological tissue, from a first location in the biological tissue to a second location in the biological tissue, the apparatus comprising:
a first external magnetic source (S1) outside of the biological tissue, the external magnetic source operative to generate a rotatable non-uniform magnetic field having an axis of rotation substantially along a line from the internal device to the external magnetic source, the non-uniform magnetic field having flux lines substantially orthogonal to the axis of rotation;
a local magnet (M1) in the internal device, the local magnet having a local magnetic field with flux lines substantially orthogonal to the axis of rotation; and
at least one helical projection on a surface of the internal device, the at least one helical projection interacting with the viscous medium to produce, from a rotation of the internal device, a rotation-based propulsive force on the internal device relative to the biological tissue;
wherein:
i. the non-uniform magnetic field of the external magnetic source (S1) is coupled to the local magnetic field of the local magnet (M1) of the internal device, such that a rotation of the non-uniform magnetic field of the external magnetic source (S1) on the axis of rotation produces a corresponding rotation of the local magnet (M1) and of the internal device;
ii. the non-uniform magnetic field has a substantial non-zero flux density gradient whose interaction with the local magnet of the internal device produces a gradient-based propulsive force substantially in a direction of the line from the internal device to the external magnetic source; and
iii. the rotation-based propulsive force combines with the gradient-based propulsive force to propel the internal device from the first location in the biological tissue towards the second location in the biological tissue.

2. The apparatus of claim 1, wherein said first external magnetic source (S1) comprises a permanent magnet, an electromagnet or a combination thereof.

3. The apparatus of claim 1, wherein the first external magnetic source (S1) is a dipole magnetic source, and wherein the local magnet (M1) of the internal device is a dipole magnet.

4. The apparatus of claim 1, wherein said first external magnetic source (S1) is a flat permanent magnet, in the form of rectangle or a flat disk, magnetized across the flat surface.

5. The apparatus of claim 2, wherein said permanent magnet comprises at least two magnets arranged in different magnetization directions, configured to maximize the magnetic flux in a specific plane and/or to maximize the magnetic gradient along a specific axis orthogonal to said plane.

6. The apparatus of claim 5, wherein said at least two magnets, are assembled in a non-planar orientation relative to each other, configured to maximize the magnetic flux in a specific plane and/or maximize the magnetic gradient along a specific axis orthogonal to said plane.

7. The apparatus of claim 6, wherein said assembly is constructed on a magnetic support structure (e.g., made of magnetic steel) to provide mechanical support to said assembly and to maximize the magnetic flux in a specific plane and/or to maximize the magnetic gradient along a specific axis orthogonal to said plane.

8. The apparatus of claim 1, wherein said local magnet (M1) is a diametrically magnetized magnet.

9. The apparatus of claim 1, wherein said internal device has a front end and a tail end and wherein local magnet (M1) is proximal to the front end of the internal device.

10. The apparatus of claim 1, wherein said internal device further comprises a magnetic component (M2), the magnetic component coupling magnetically with the non-uniform magnetic field of the external magnetic source (S1).

11. The apparatus of claim 10, wherein said magnetic component (M2) is axially magnetized along the axis of the helical projection.

12. The apparatus of claim 10, further comprising a second external magnetic source (S2), said second source comprises a permanent magnet.

13. The apparatus of claim 12, wherein said magnetic component (M2), coupling magnetically with said second external source (S2).

14. The apparatus of claim 10, wherein the magnetic component (M2) of the internal device comprises a magnetizable material, wherein the magnetizable material is coupled magnetically to the external first magnetic source, second magnetic source or a combination thereof.

15. The apparatus of claim 10, wherein the magnetic component (M2) of the internal device comprises a permanent magnet with a magnetic field having flux lines substantially parallel to the line from the internal device to the external magnetic source.

16. The apparatus of claim 10,
wherein the non-uniform magnetic field (the field of S1) has a reversible flux direction; and
wherein:
i. when the flux direction of the non-uniform magnetic field is a first direction along the line from the internal device to the external magnetic source, then the gradient-based propulsive force attracts the internal device toward the external magnetic source; and
ii. when the flux direction of the non-uniform magnetic field is a second direction along the line from the internal device to the external magnetic source, then the gradient-based propulsive force repels the internal device away from the external magnetic source.

17. The apparatus of claim 10, wherein the permanent magnet (M2) comprises a spherical magnet (M2) that is free to rotate within a cavity in the internal device.

18. The apparatus of claim 10, wherein the magnet (M2) is diametrically magnetized orthogonal to the symmetry axis of the internal device, and magnet (M2) is free to rotate inside a cavity in the internal device.

19. The apparatus of claim 18, wherein the diametrically magnetized magnet (M2) is a cylinder and is not affixed to the cavity in the internal device, and wherein the cavity is a cylindrical cavity of the same dimensions as M2.

20. The apparatus of claim 12, wherein the local magnet (M1) is partially enclosed within a magnetic shielding material.

21. The apparatus of claim 20, wherein the magnetic shielding material is removable and replaceable.

22. A method of propelling an internal device within a viscous medium in a biological tissue, from a first location in the biological tissue to a second location in the biological tissue, the method comprising:
a) providing (i) the internal device at the first location in the biological tissue and (ii) a first external magnetic source (S1) outside of the biological tissue;
wherein the internal device has an axis of rotation and comprises a local magnet (M1) having a local magnetic field with flux lines substantially orthogonal to the axis of rotation and at least one helical projection on a surface;
b) with the first external magnetic source (S1), generating a rotating non-uniform magnetic field having an axis of rotation substantially along a line from the internal device to the external magnetic source, the non-uniform magnetic field having flux lines substantially orthogonal to the axis of rotation; a local magnet (M1) in the internal device;
wherein rotation of the rotating non-uniform magnetic field of the external magnetic source (S1) on the axis of rotation produces a corresponding rotation of the local magnet (M1) and of the internal device, and from the rotation of the internal device the at least one helical projection interacts with the viscous medium to produce, a rotation-based propulsive force on the internal device relative to the biological tissue; and
wherein the rotating non-uniform magnetic field has a substantial non-zero flux density gradient whose interaction with the local magnet of the internal device produces a gradient-based propulsive force substantially in a direction of the line from the internal device to the external magnetic source; and
wherein the rotation-based propulsive force combines with the gradient-based propulsive force to propel the internal device from the first location in the biological tissue towards the second location in the biological tissue; and
c) adjusting the rotating non-uniform magnetic field to propel the internal device from the first location to the second location in the biological tissue.

23. The method of claim 22, wherein the first external magnetic source (S1) comprises a permanent magnet, an electromagnet or a combination thereof.

24. The method of claim 22, wherein the first external magnetic source (S1) is a dipole magnetic source, and wherein the local magnet (M1) of the internal device is a dipole magnet.

25. The method of claim 22, wherein the first external magnetic source (S1) is a flat permanent magnet, in the form of rectangle or a flat disk, magnetized across the flat surface.

26. The method of claim 23, where said permanent magnet comprises at least two magnets arranged in different magnetization directions, configured to maximize the magnetic flux in a specific plane and/or to maximize the magnetic gradient along a specific axis orthogonal to said plane.

27. The method of claim 26, wherein said at least two magnets, are assembled in a non-planar orientation relative to each other, configured to maximize the magnetic flux in a specific plane and/or maximize the magnetic gradient along a specific axis orthogonal to said plane.

28. The method of claim 27, wherein said assembly is constructed on a magnetic support structure (e.g., made of magnetic steel) to provide mechanical support to said assembly and to maximize the magnetic flux in a specific plane and/or to maximize the magnetic gradient along a specific axis orthogonal to said plane.

29. The method of claim 22, wherein the local magnet (M1) is a diametrically magnetized magnet.

30. The method of claim 22, wherein said internal device has a front end and a tail end and wherein local magnet (M1) is proximal to the front end of the internal device.

31. The method of claim 22, wherein the internal device further comprises a magnetic component (M2), the magnetic component coupling magnetically with the rotating non-uniform magnetic field of the external magnetic source (S1).

32. The method of claim 31, wherein said magnetic component (M2) is axially magnetized along the axis of the helical projection.

33. The method of claim 31, further comprising providing a second external magnetic source (S2) outside of the biological tissue, said second source comprises a permanent magnet.

34. The method of claim 33, wherein said magnetic component (M2), coupling magnetically with said second external source (S2).

35. The method of claim 31, wherein the magnetic component (M2) of the internal device comprises a magnetizable material, wherein the magnetizable material is coupled magnetically to the external first magnetic source, second magnetic source or a combination thereof.

36. The method of claim 31, wherein the magnetic component (M2) of the internal device comprises a permanent magnet with a magnetic field having flux lines substantially parallel to the line from the internal device to the external magnetic source.

37. The method of claim 31,
wherein the non-uniform magnetic field (the field of S1) has a reversible flux direction; and
wherein:
i. when the flux direction of the non-uniform magnetic field is a first direction along the line from the internal device to the external magnetic source, then the gradient-based propulsive force attracts the internal device toward the external magnetic source; and
ii. when the flux direction of the non-uniform magnetic field is a second direction along the line from the internal device to the external magnetic source, then the gradient-based propulsive force repels the internal device away from the external magnetic source.

38. The method of claim 31, wherein the permanent magnet (M2) comprises a spherical magnet (M2) that is free to rotate within a cavity in the internal device.

39. The method of claim 31, wherein the magnet (M2) is diametrically magnetized orthogonal to the symmetry axis of the internal device, and magnet (M2) is free to rotate inside a cavity in the internal device.

40. The method of claim 39, wherein the diametrically magnetized magnet (M2) is a cylinder and is not affixed to the cavity in the internal device, and wherein the cavity is a cylindrical cavity of the same dimensions as M2.

41. The method of claim 33, wherein the local magnet (M1) is partially enclosed within a magnetic shielding material.

42. The method of claim 41, wherein the magnetic shielding material is removable and replaceable.
